(19) United States
(12) Patent Application Publication
(10) Pub. No.: US 2009/0048466 A1
(43) Pub. Date: Feb. 19, 2009

(54) PROCESS FOR PREPARING AMINES BY CONDITIONING THE CATALYST WITH AMMONIA

(75) Inventors: Christian Lettmann, Coesfeld (DE); Juergen Lippe, Gelsenkirchen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: EVONIK DEGUSSA GmbH, Essen (DE)

(21) Appl. No.: 12/278,795

(22) PCT Filed: Nov. 29, 2006
(86) PCT No.: PCT/EP2006/069056

§ 371 Date: Aug. 8, 2008

(30) Foreign Application Priority Data
Feb. 14, 2006 (DE) 10 2006 006 625.1

(51) Int. Cl. C07C 209/48; B01J 25/00

(52) U.S. Cl. 564/448; 502/301

(57) ABSTRACT

The invention relates to a process for preparing amines by conditioning the catalyst with ammonia.

The invention relates to a process for preparing amines by conditioning the catalyst with ammonia.

The preparation of amines and diamines by means of catalytic hydrogenation of the corresponding nitrites or by catalytic reductive amination of the aldehydes or ketones is known. Suitable examples are nickel catalysts, copper catalysts, iron catalysts, palladium catalysts, rhodium catalysts, ruthenium catalysts and cobalt catalysts.

For many applications, cobalt catalysts and ruthenium catalysts are preferred, since they have a high selectivity with respect to the formation of primary amines (cf., for example, Jiri Volf and Josef Pasek, “Hydrogenation of Nitriles”, Studies in Surface Science and Catalysis, 27 (1986) 105-144; Silvia Gomez et al., “The Reductive Amination of Aldehydes and Ketones and the Hydrogenation of Nitriles: Mechanistic Aspects and Selectivity Control, Adv. Synth. Catal. 344 (2003) 1037-1057).

Numerous methods have been described for increasing the yield of primary amine in nitrile hydrogenations or reductive aminations.

U.S. Pat. No. 6,521,564 (Roche Vitamins, Inc.) describes a process for modifying nickel and cobalt catalysts. Before their first use, the catalysts are treated with a modifier. Examples of suitable modifiers are carbon monoxide, carbon dioxide, aldehydes and ketones. The catalysts are suspended in a solvent, treated with the modifier, removed from the solution, washed repeatedly and then used to hydrogenate nitrites. The catalysts thus modified have a higher selectivity with respect to the formation of the primary amine than unmodified catalysts. A disadvantage of this process is the relatively complicated modification which necessitates additional process steps. In addition, there is the risk that the modifiers are partly released again during the hydrogenation process and hence adversely affect the product purity.

The modification with alkali metal hydroxides (U.S. Pat. No. 4,375,003), especially lithium hydroxide (EP 0 913 388), likewise leads to an improvement in the yield of primary amine. The catalysts can either be treated with alkali metal hydroxides before the reaction, or else the alkali metal hydroxide is added to the reaction mixture during the reaction. Provided that no relatively large amounts of solvents such as ammonia, THF or methanol are used, the long-term stability of the LiOH-modified catalysts is quite good. In in-house experiments, however, we found that, when abovementioned solvents are used, the LiOH is washed continuously from the catalyst and the proportion of secondary amines thus raises again. In a continuous process in which the solvent is removed from the mixture by distillation and recycled into the process, there is additionally deposition of the alkali metal hydroxides in the distillation columns. The columns have to be shut down at regular intervals and cleaned, so that the alkali modification leads indirectly to production shutdowns.

According to EP 0 913 387, quaternary ammonium bases can also be used to increase the selectivity. Especially in the case of use of a solvent, correspondingly modified catalysts have a significantly higher lifetime than alkali-modified catalysts. A crucial disadvantage is the relatively high cost of quaternary ammonium bases.

It has been stated many times that the addition of ammonia to the reaction mixture or the use of ammonia as a solvent in nitrile hydrogenations leads to an increase in the yield of primary amine. The same applies to reductive aminations, in which an excess of ammonia or the use of ammonia as a solvent likewise has a positive effect on the yield (for example EP 449 089, EP 659 734, DE 12 29 078).

The positive influence of ammonia on the selectivity is frequently explained by the following reaction scheme (see, for example, the reviews by: Jiri Volf and Josef Pasek, “Hydrogenation of Nitriles”, Studies in Surface Science and Catalysis, 27 (1986) 105-144; Silvia Gomez et al., “The Reductive Amination of Aldehydes and Ketones and the Hydrogenation of Nitriles: Mechanistic Aspects and Selectivity Control, Adv. Synth. Catal. 344 (2003) 1037-1057):

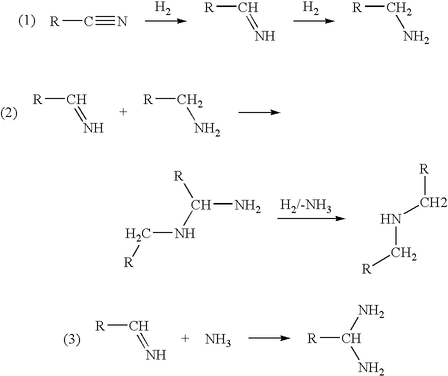

In the nitrile hydrogenation, according to (1), a hydrogen molecule is first added on to form an intermediate imine. For the imine, which also occurs as an intermediate in the reductive amination, there are several possible reactions. The addition of a further molecule of hydrogen according to (1) leads to the desired product, the primary amine. However, according to (2), an already formed primary amine can also add on to the imine, which leads to the formation of the undesired secondary amine in the subsequent reaction steps. This secondary amine can in turn react by addition onto an imine and subsequent elimination/hydrogenation to give the tertiary amine (not shown). The addition of ammonia to the reaction mixture leads to an increase in selectivity because ammonia is added on to the imine according to (3) and thus suppresses the reaction of the imine with other amines. The subsequent hydrogenation of the gem-diamine leads to the target product, the primary amine. One disadvantage of the addition of ammonia to the reaction mixture is the reduction in the catalyst activity (see, for example, U.S. Pat. No. 4,375,003 Example IX; C.D. Frohning in: J. Falbe and U. Hasserodt (Eds.) “Katalysatoren, Tenside und Mineralöladditive” [Catalysts, Surfactants and Mineral Oil Additives], Georg Thieme verlag Stuttgart, 1978, p. 44 ff.; Jiri Volf and Josef Pasek, “Hydrogenation of Nitriles”, Studies in Surface Science and Catalysis, 27 (1986) 105-144).

It has now been found that, surprisingly, the selectivity increase which is achieved by the addition of ammonia to the reaction mixture can be enhanced significantly when the catalyst is additionally treated with ammonia (conditioning) before it is used in the hydrogenation and only then is contacted with the reaction mixture composed of hydrogen, starting compounds and ammonia. The conditioning of the catalyst with ammonia also has the advantage that the catalyst has a significantly higher activity than without conditioning.

The invention provides a process for preparing amines, diamines or polyamines by means of catalytic hydrogenation and/or by catalytic reductive amination of the corresponding starting compounds in the presence of ammonia, hydrogen and of at least one catalyst and optionally of a solvent or solvent mixture, wherein the catalyst is treated (conditioned) with ammonia before the start of the hydrogenation or reductive amination.

The treatment (conditioning) of the catalyst can be carried out with gaseous, liquid or supercritical ammonia. When a solvent is used, the conditioning can also be effected with a mixture of the solvent(s) with ammonia. In a preferred embodiment, the conditioning is effected exclusively using liquid ammonia.

The conditioning can be carried out either at the pressure which arises from the vapour pressure of the ammonia at the appropriate conditioning temperature, or at elevated pressure of from 50 to 300 bar, preferably from 200 to 250 bar, is employed. The pressure increase can quite generally be achieved by gases such as nitrogen, argon, and/or hydrogen. The only upper limit on the maximum employable pressure is the pressure resistance of the apparatus used. In a preferred embodiment, the conditioning with ammonia is effected additionally in the presence of hydrogen. The partial pressure of the hydrogen used in the reactor is in the range from 0.1 to 300 bar, preferably from 50 to 250 bar, more preferably from 100 to 200 bar. Higher pressures than those specified above have no adverse effects.

The conditioning can be carried out within a wide temperature range. Typically, temperatures between 20 and 180° C., preferably from 50 to 130° C., are employed. Particular preference is given to passing through a temperature ramp in which the catalyst, beginning at moderately elevated temperature, preferably between 20 and 50° C., is heated slowly to the reaction temperature desired later for the hydrogenation, preferably from 50 to 130° C.

In reactors which have only a low pressure resistance, it may, though, be advantageous to work at temperatures below room temperature in order to lower the vapour pressure of the ammonia correspondingly.

The conditioning can in principle be effected actually before the catalyst is introduced into the reactor. Especially in the case of fixed bed catalysts, it is, though, advantageous when the treatment of the catalyst is effected with ammonia only after the catalyst has been introduced into the reactor. One means of this is to flood the fixed bed reactors with ammonia after the catalyst has been introduced, so that the entire amount of catalyst comes into contact with ammonia. However, preference is given to continuous treatment with ammonia in which a constant ammonia stream through the reactor is preferably established. The amount of ammonia in this context is between 0.2 and 3 m³, preferably 0.5 and 2 m³ of ammonia per m³ of catalyst and hour. To minimize the ammonia consumption, ammonia arriving at the reactor outlet can be recycled back to the reactor inlet either directly or after preceding purification, preferably distillation. The duration of the conditioning is dependent upon the amount of ammonia used and is preferably between 1 and 48 hours, more preferably between 12 and 24 hours. Longer periods do not adversely affect the result and are likewise possible in the context of the invention. It is preferred that the conditioning is continued at least until the entire catalyst has been saturated with ammonia, i.e., for example, in the case of a porous catalyst, virtually the entire pore volume should be filled with ammonia.

The catalysts used may in principle be all catalysts which catalyse the hydrogenation of nitrile and/or imine groups with hydrogen. Particularly suitable catalysts are nickel catalysts, copper catalysts, iron catalysts, palladium catalysts, rhodium catalysts, ruthenium catalysts and cobalt catalysts, very particularly cobalt catalysts. To increase the activity, selectivity and/or lifetime, the catalysts may additionally comprise dopant metals or other modifiers. Typical dopant metals are, for example, Mo, Fe, Ag, Cr, Ni, V, Ga, In, Bi, Ti, Zr and Mn, and also the rare earths. Typical modifiers are, for example, those with which the acid-based properties of the catalysts can be influenced, for example alkali metals and alkaline earth metals or compounds thereof, preferably Mg and Ca compounds, and also phosphoric acid or sulphuric acid and compounds thereof.

The catalysts may be used in the form of powders or shaped bodies, for example extrudates or compressed powders. It is possible to use unsupported catalysts, Raney-type catalysts or supported catalysts. Preference is given to Raney-type and supported catalysts. Suitable support materials are, for example, kieselguhr, silicon dioxide, aluminium oxide, alumosilicates, titanium dioxide, zirconium dioxide, aluminium-silicon mixed oxides, magnesium oxide and activated carbon. The active metal may be applied to the support material in the manner known to the person skilled in the art, for example by impregnation, spraying or precipitation. Depending on the type of catalyst preparation, further preparation steps known to those skilled in the art are necessary, for example drying, calcining, shaping and activation. For the shaping, further assistants, for example graphite or magnesium stearate, may optionally be added.

Preference is given to using catalysts as obtainable according to the teachings of EP 1 207 149 (catalysts based on an activated, alpha-Al₂O₃-containing Raney catalyst having macropores and based on an alloy of aluminium and at least one transition metal selected from the group consisting of iron, cobalt and nickel and optionally one or more further transition metals selected from the group consisting of titanium, zirconium, chromium and manganese), EP 1 207 149, EP 1 209 146, U.S. Pat. No. 3,558,709, EP 880 996, EP 623 585, EP 771 784, EP 814 098 (a catalyst which comprises, as the active metal, ruthenium alone or together with at least one metal of transition group I, VII or VIII of the Periodic Table applied to a support in an amount of from 0.01 to 30% by weight based on the total weight of the catalyst, from 10 to 50% by weight of the pore volume of the support being formed by macropores having a pore diameter in the range from 50 nm to 10 000 nm and from 50 to 90% of the pore volume of the support being formed by mesopores having a pore diameter in the range of from 2 to 50 nm, the sum of the pore volumes adding up to 100%), EP 636 409 (cobalt catalysts whose catalytically active composition comprises from 55 to 98% by weight of cobalt, from 0.2 to 15% by weight of phosphorus, from 0.2 to 15% by weight of manganese and from 0.2 to 15% by weight of alkali metal, calculated as oxide, characterized in that the catalyst mass is calcined in a first step at end temperatures of from 550 to 750° C. and in a second step at end temperatures of from 800 to 1000° C.), EP 1 221 437, WO 97/10202 and EP 813 906 (a catalyst which comprises, as the active metal, ruthenium alone or together with at least one metal of transition group I, VII or VIII of the Periodic Table, applied to a support, the support having a mean pore diameter of at least 50 nm and a BET surface area of at most 30 m²/g, and the amount of the active metal being from 0.01 to 30% by weight, based on the total weight of the catalyst, the ratio of the surface areas of the active metal and of the catalyst being <0.05).

Particular preference is given to shaped catalysts as obtainable according to EP 1 216 985 (shaped hydrogenation catalyst based on Raney cobalt is used, which is characterized in that the Raney catalyst is present in the form of hollow bodies). Particular preference is likewise given to supported cobalt catalysts as described in EP 1 306 365, in which the crystals of the cobalt and of any nickel present have a mean particle size of from 3 to 30 nm.

The process according to the invention is found to be advantageous in the conversion of nitrites and the reductive aminations of ketones and aldehydes as starting compounds, since the yield of primary amine is significantly increased by the treatment of the catalyst with ammonia. The corresponding starting compounds which are suitable for the process will now be described. Suitable starting compounds are mono-, di- and polynitriles, iminonitriles and aminonitriles. It is also possible to convert compounds which contain one or more nitrile, imine and/or amine groups and simultaneously one or more aldehyde and/or keto groups. It is also possible to convert mono-, di- or polyaldehydes or mono-, di- or polyketones and compounds which contain one or more aldehyde groups and simultaneously one or more keto groups.

Furthermore, the process according to the invention is also suited for the conversion of polyetherpolyols with ammonia to the corresponding polyetheramines.

Preference is given to using cyclic compounds of the general formula (I)

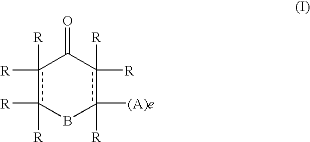

A=CN, CH₃;
e=0, 1;
B=C═O, CH₂, CHR, CR₂, NH, NR, O;
R=simultaneously or each independently, hydrogen, branched or unbranched aryl, alkyl, alkenyl, alkynyl and cycloalkyl radicals, which may be substituted or unsubstituted, and N-, O-, S- or P-containing substituents which are bonded directly to the ring via a heteroatom or carbon atom, and where no, one or two double bonds may be present.

Examples of such compounds are:

| | | | |
|---|---|---|---|
| 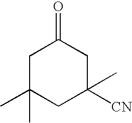 | Isophoronenitrile(IPN) | 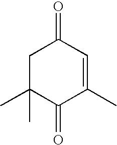 | Oxoisophorone |
| 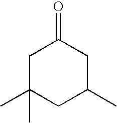 | 3,5,5-Trimethylcyclohexanone(TMC-one) | 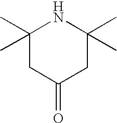 | 2,2,6,6-Tetramethyl-4-piperidone |
| 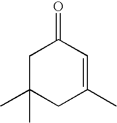 | Isophorone | | |
| 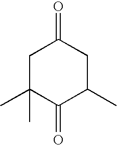 | 3,5,5-Trimethylcyclo-hexane-1,4-dione | | |

Likewise suitable is 1,3- and 1,4-cyclohexanedialdehyde and mixtures thereof, of the formula

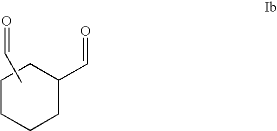

Particular preference is given to isophoronenitrile and 3,5,5-trimethylcyclohexane-1,4-dione.

Preferred aromatic compounds are those of the general formula (II)

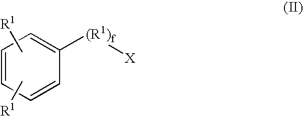

where the symbols are each defined as follows:
X: cyanide (CN), aldehyde (CHO) or keto group (CR¹O);
R¹: simultaneously or each independently, hydrogen, branched or unbranched aryl, alkyl, alkenyl, alkynyl and cycloalkyl radicals, which may be substituted or unsubstituted, and N-, O-, S- or P-containing substituents which are bonded directly to the aromatic ring via a heteroatom or carbon atom;
f: values from 0 to 5.

Preferred examples thereof are:

| | | | |
|---|---|---|---|
| 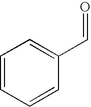 | Benzaldehyde | 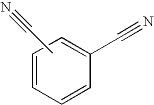 | Phthalonitrile,terephthalonitrile,isophthalonitrileand mixtures thereof |
| 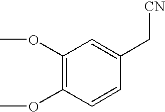 | 3,4-Dimethoxy-phenylacetonitrile | | |

Preferred linear or branched compounds are those of the formula (III)

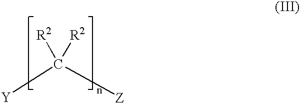

where the symbols are each defined as follows:
n: integers from 0 to 18;
Y, Z: simultaneously or each independently, cyanide (CN), aldehyde (CH), ketone (CR²⁰), hydrogen, CH₃, CR═CR² or amines (CNR₂²),
R²: simultaneously or each independently, hydrogen, branched or unbranched aryl, alkyl, alkenyl alkynyl and cycloalkyl radicals, which may be substituted or unsubstituted, and N—, O—, S— or P— containing substituents.

Preferred examples thereof are:

| | |
|---|---|
| 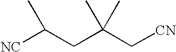 | 2,4,4- and 2,2,4-Trimethyladiponitrileand mixtures thereof |
| 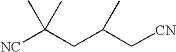 | |
| 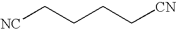 | Adiponitrile |
| 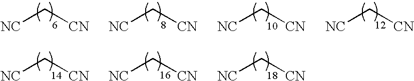 | Fatty nitriles (alone or in mixtures) |
| 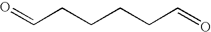 | 1,6-Hexanedial |
| 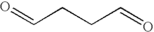 | 1,4-Butanedial |
| 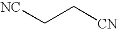 | 1,4-Butanedinitrile |
| 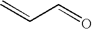 | Acrolein |
| 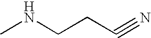 | 3-Methylaminopropanenitrile |
| 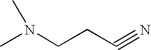 | 3-Dimethylaminopropanenitrile |
| 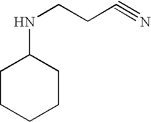 | 3-Cyclohexylaminopropanenitrile |
| 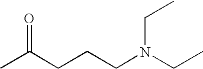 | 1-Diethylaminopentan-4-one |

For the conversion of polyetherpolyols with ammonia to the corresponding polyetheramines, polyetherpolyols with molecular weights between 200 and 5000 g/mol are suited. Preferred examples thereof are:

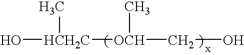

Polyoxypropylenediols, with x=2-70

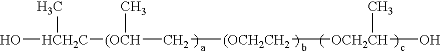

Poly(oxyethylen-oxypropylene)diols, a+c=2-6; b=2-40

In the process according to the invention, very particular preference is given to using isophorone-nitrile, trimethyladiponitrile, adiponitrile, isophoroneiminonitrile and isophoroneaminonitrile.

The invention is illustrated in detail by the examples which follow.

### EXAMPLES

### Example 1

### Aminating hydrogenation of 3-cyano-3,5,5-trimethyl-cyclohexanone (isophoronenitrile) to 3-amino-methyl-3,5,5-trimethylcyclohexylamine (isophorone-diamine) with a supported cobalt catalyst

The experimental apparatus consisted of a 100 ml fixed bed reactor charged with ion exchanger according to EP 042 119 for catalysing the imine formation from IPN and ammonia, and a downstream fixed bed reactor charged with 300 ml of a tabletted cobalt catalyst (kieselguhr support). To condition the catalyst, 300 ml/h (180 g/h) of ammonia were passed over the fixed bed at temperatures between 60 and 100° C. During the conditioning, a partial hydrogen pressure of approx. 100 bar was established. After two hours, the conditioning had ended. Directly after the conditioning, 30 ml/h (approx. 28 g/h) of IPN and 400 ml/h (370 g/h) of ammonia were fed in. The two reactant streams were mixed immediately upstream of the reactor charged with ion exchanger. The result of the gas chromatography analysis of the product is listed in Table 1 below in the “with conditioning” column.

### Comparative Example A

The experiment was carried out with the same amount of fresh catalyst, but this time without ammonia conditioning (see Table 1, “without conditioning” column).

TABLE 1

| | | Without |
|---|---|---|
| | With | conditioning |
| | conditioning | Comparative |
| Analysis result (GC %) | Example 1 | Example A |
| Total isophoronediamine | 88.4 | 83.7 |
| Total isophoroneaminonitrile | 1.5 | 6.2 |
| Imine (2-aza-4,6,6-trimethyl- | 5.3 | 5.7 |
| bicyclo[3.2.1]octane) | | |
| Amidine (3,3,5-trimethyl- | 0.7 | 2.3 |
| 6-amino-7-aza- | | |
| bicyclo[3.2.1]octane) | | |
| Others | 4.1 | 2.1 |

With conditioning, the diamine yield is significantly higher than without conditioning. This is attributable firstly to the higher conversion (less isophorone-aminonitrile intermediate in the product) and secondly to the higher selectivity (lower proportion of the cyclic amidine and imine by-products).

### Example 2

### Aminating hydrogenation of 3-cyano-3,5,5-trimethylcyclohexanone (isophoronenitrile) to 3-aminomethyl-3,5,5-trimethylcyclohexylamine (isophoronediamine) with a Raney-type cobalt catalyst

The experimental apparatus consisted of a 100 ml fixed bed reactor charged with ion exchanger according to EP 042 119 to catalyse the imine formation from IPN and ammonia, and a downstream fixed bed reactor charged with 66 ml of a spherical Raney-type cobalt catalyst. To condition the catalyst, 100 ml/h (60 g/h) of ammonia were passed over the fixed bed at approx. 100° C. During the conditioning, a partial hydrogen pressure of approx. 100 bar was established. After two hours, the conditioning had ended. Directly after the conditioning, 100 ml/h of a 14% solution of IPN in ammonia were fed in (LHSV=1.5 h⁻¹). The result of the gas chromatography analysis of the product is listed in Table 2 below in the “with conditioning” column.

### Comparative Example B

The experiment was carried out with the same amount of fresh catalyst, but this time without the ammonia conditioning (see Table 2, “without conditioning” column).

TABLE 2

| | | Without |
|---|---|---|
| | With | conditioning |
| | conditioning | Comparative |
| Analysis result (GC %) | Example 2 | Example B |
| Total isophoronediamine | 94 | 91 |
| Total isophoroneaminonitrile | 0.2 | 2.6 |
| Imine (2-aza-4,6,6-trimethyl- | 2.3 | 1.6 |
| bicyclo[3.2.1]octane) | | |
| Amidine (3,3,5-trimethyl- | 2 | 3.2 |
| 6-amino-7-aza- | | |
| bicyclo[3.2.1]octane) | | |
| Others | 1.5 | 1.5 |

With conditioning, the diamine yield is 3% higher than without conditioning. This is attributable firstly to the higher conversion (less isophoroneaminonitrile intermediate in the product) and secondly to the higher selectivity (lower proportion of the cyclic amidine and imine by-products).

### Example 3

### Hydrogenation of Trimethylhexamethylenedinitrile to Trimethylhexamethylenediamine

The experiments were carried out in a 1 l hydrogenation autoclave which was equipped with a catalyst basket (static basket with stirrer, Mahoney type). The catalyst basket was in each case charged freshly with 80 ml of a spherical Raney-type fixed bed cobalt catalyst.

For conditioning with ammonia, the reactor was first charged with approx. 500 ml of ammonia and kept at 50° C. and 250 bar with stirring for approx. 2 h. The ammonia was then discharged by decompressing the reactor.

After the conditioning, 600 ml of a 20% solution of trimethylhexamethylenedinitrile (mixture of the 2,4,4- and 2,2,4-isomers) were hydrogenated at 120° C. and total pressure 250 bar for 6 h. The product was discharged and subsequently analysed by gas chromatography. The result is listed in Table 3 in the “with conditioning” column.

### Comparative Example C

In the same way, an experiment was carried out in which the catalyst had not been conditioned beforehand with ammonia. The result is listed in Table 3 in the “without conditioning” column.

TABLE 3

| | | Without |
|---|---|---|
| | With | conditioning |
| | conditioning | Comparative |
| Analysis result (GC %) | Example 3 | Example C |
| Total trimethylhexamethylene- | 90 | 84 |
| diamine | | |
| Imine | 4.8 | 6.2 |
| Saturated cyclic compounds | 1.8 | 2.6 |
| Hydrogenatable intermediates | 0.1 | 0.11 |
| Further by-products | 3.3 | 7 |

The conditioning of the catalyst with ammonia leads to a reduction in by-product formation with unchanged activity.

1. A process for preparing amines, diamines or polyamines by means of catalytic hydrogenation and/or by catalytic reductive amination of the corresponding starting compounds in the presence of ammonia, hydrogen and of at least one catalyst and optionally of a solvent or solvent mixture,
characterized in that
the catalyst is treated (conditioned) with ammonia before the start of the hydrogenation or reductive amination.

2. A process according to claim 1,
characterized in that
the treatment (conditioning) of the catalyst is carried out with gaseous, liquid and/or supercritical ammonia.

3. A process according to claim 1,
characterized in that
at least one solvent is used.

4. A process according to claim 1,
characterized in that
the conditioning is effected using liquid ammonia.

5. A process according to claim 1,
characterized in that
the conditioning is carried out at the pressure which arises from the vapour pressure of the ammonia at the appropriate conditioning temperature.

6. A process according to claim 1,
characterized in that
operation is effected at elevated pressure of from 50 to 300 bar.

7. A process according to claim 1,
characterized in that
the conditioning is additionally effected by increasing the pressure by means of gases, preferably nitrogen, argon and/or hydrogen.

8. A process according to claim 7,
characterized in that
the partial pressure of the hydrogen in the reactor is in the range from 0.1 to 300 bar.

9. A process according to claims claim 1,
characterized in that
temperatures between 20 and 180° C. are employed.

10. A process according to claim 1,
characterized in that
a temperature ramp is passed through, in which the catalyst, beginning at moderately elevated temperature, is heated slowly to the reaction temperature desired later for the hydrogenation.

11. A process according to claim 1,
characterized in that
the conditioning is effected before the catalyst is introduced into the reactor.

12. A process according to claim 1,
characterized in that
the catalyst is conditioned with ammonia after the catalyst has been introduced into the reactor.

13. A process according to claim 1,
characterized in that
the catalyst is conditioned by means of a continuous ammonia stream.

14. A process according to claim 1,
characterized in that
the conditioning is effected for between 1 and 48 hours.

15. A process according to claim 1,
characterized in that
the conditioning is continued until the entire amount of catalyst has been saturated with ammonia.

16. A process according to claim 1,
characterized in that
nickel catalysts, copper catalysts, iron catalysts, palladium catalysts, rhodium catalysts, ruthenium catalysts and cobalt catalysts are used.

17. A process according to claim 1,
characterized in that
dopant metals are present in the catalysts.

18. A process according to claim 1,
characterized in that
unsupported catalysts, Raney-type catalysts or supported catalysts are used.

19. A process according to claim 18,
characterized in that
the support material used is kieselguhr, silicon dioxide, aluminium oxide, alumosilicates, titanium dioxide, zirconium dioxide, aluminium-silicon mixed oxides, magnesium oxide and activated carbon.

20. A process according to claim 1,
characterized in that
fixed bed catalysts are used.

21. A process according to claim 20,
characterized in that
the fixed bed reactors, after the catalyst has been introduced, are flooded with ammonia, such that the entire amount of catalyst comes into contact with ammonia.

22. A process according to claim 1,
characterized in that
the starting compounds used are mono-, di- and polynitriles, iminonitriles or aminonitriles.

23. A process according to claim 1,
characterized in that
the starting compounds used are mono-, di- or polyaldehydes or mono-, di- or polyketones.

24. A process according to claim 1,
characterized in that
the starting compounds used are compounds which contain one or more nitrile, imine and/or amine groups and simultaneously one or more aldehyde and/or keto groups.

25. A process according to claim 1,
characterized in that
the starting compounds used are compounds which contain one or more aldehyde groups and simultaneously one or more keto groups.

26. A process according to claim 1,
characterized in that
the starting compounds used are isophoronenitrile (IPN), 3,5,5-trimethylcyclohexanone (TMC-one), isophorone, 3,5,5-trimethylcyclohexane-1,4-dione, oxoisophorone, 2,2,6,6-tetramethyl-4-piperidone and 1,3- and 1,4-cylohexanedialdehyde, and mixtures thereof.

27. A process according to claim 1,
characterized in that
the starting compounds used are benzaldehyde, 3,4 dimethoxyphenylacetonitrile and phthalonitrile, terephthalonitrile, isophthalonitrile and mixtures thereof.

28. A process according to claim 1,
characterized in that
the starting compounds used are 2,4,4- and 2,2,4-trimethyladiponitrile and mixtures thereof, adiponitrile, 1,6-hexanedial, 1,4-butanedial, 1,4-butanedinitrile, 3-methylaminopropanenitrile, 3-dimethylaminopropanenitrile, 3-cyclohexylaminopropanenitrile, 1-diethylaminopentan-4-one and acrolein.

29. A process according to claim 1,
characterized in that
isophoronenitrile, trimethyladiponitrile, adiponitrile, isophoroneiminonitrile and isophoroneaminonitrile are used.

30. A process according to claim 1,
characterized in that
polyetherpolyols are used.

31. A process according to claim 1,
characterized in that
catalysts in which the crystals of the cobalt and of any nickel present have a mean particle size of from 3 to 30 nm are used.

32. A process according to claim 1,
characterized in that
a catalyst which comprises, as the active metal, ruthenium alone or together with at least one metal of transition group I, VII or VIII of the Periodic Table applied to a support in an amount of from 0.01 to 30% by weight based on the total weight of the catalyst, from 10 to 50% by weight of the pore volume of the support being formed by macropores having a pore diameter in the range from 50 nm to 10 000 nm and from 50 to 90% of the pore volume of the support being formed by mesopores having a pore diameter in the range from 2 to 50 nm, the sum of the pore volumes adding up to 100%, is used.

33. A process according to claim 1,
in the presence of a catalyst which comprises, as the active metal, ruthenium alone or together with at least one metal of transition group I, VII or VIII of the Periodic Table, applied to a support, the support having a mean pore diameter of at least 50 nm and a BET surface area of at most 30 m2/g, and the amount of the active metal being from 0.01 to 30% by weight, based on the total weight of the catalyst, the ratio of the surface areas of the active metal and of the catalyst support being <0.05.

34. A process according to claim 1,
characterized in that
a shaped hydrogenation catalyst based on Raney cobalt is used, which is characterized in that the Raney catalyst is present in the form of hollow bodies.

35. A process according to claim 1,
characterized in that
cobalt catalysts are used whose catalytically active composition comprises from 55 to 98% by weight of cobalt, from 0.2 to 15% by weight of phosphorus, from 0.2 to 15% by weight of manganese and from 0.2 to 15% by weight of alkali metal, calculated as oxide,
characterized in that the catalyst mass is calcined in a first step at end temperatures of from 550 to 750° C. and in a second step at end temperatures of from 800 to 1000° C.

36. A catalyst according to claim 1,
characterized in that
catalysts are used which are based on an activated, alpha-Al2O3-containing Raney catalyst having macropores and based on an alloy of aluminium and at least one transition metal selected from the group consisting of iron, cobalt and nickel and optionally one or more
further transition metals selected from the group consisting of titanium, zirconium, chromium and manganese.
